(19) United States
(12) Patent Application Publication
(10) Pub. No.: US 2003/0095930 A1
(43) Pub. Date: May 22, 2003

(54) Composition for oral hygiene

(76) Inventors: Luiz Bellino Simionato, Sao Paulo (BR); Maria Cristina Guedes Jorge Micheletto, Sao Paulo (BR); Wagner Soares Ramos, Sao Paulo (BR)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/122,977

(22) Filed: Apr. 15, 2002

(30) Foreign Application Priority Data
Apr. 26, 2001 (BR) PI 0101911-2

(51) Int. Cl. A61K 7/16

(52) U.S. Cl. 424/49

(57) ABSTRACT

Composition for oral hygiene, comprising an aqueous vehicle carrying an efficient quantity of a germicidal active agent for oral hygiene, which is adsorbed in particles with 80-100 nm of a water insoluble non-toxic polymer, in a quantity of about 1% to 20% by weight of said particles, said polymer presenting a spongy

### FIELD OF THE INVENTION

The present invention refers to a composition for oral hygiene, of the type comprising a rinsing liquid vehicle carrying one or more active agents, such as cleaning agents, flavoring agents, germicidal agents, and others.

### BACKGROUND OF THE INVENTION

There are known in the state of the art different rinsing or mouth washing compositions for providing oral hygiene with germicidal effects, either separately or together with a tooth brushing operation.

One of the deficiencies presented by the known compositions result from the fact that the active agents, especially those with bactericidal or germicidal functions, are retained in the oral cavity only for a short period of time. The capacity of actuation of these active agents, which are prematurely dissolved and carried by the saliva, is considerably limited in spite of the benefits of the cleansing effect achieved during their permanence in the oral cavity.

Aiming at minimizing the deficiency mentioned above, U.S. Pat. No. 3,394,001 proposed a composition for oral hygiene presenting a longer germicidal effect than that obtained by other water soluble compositions. In order to increase the time of actuation of the active agents, the composition proposed in said patent, conventionally comprising a liquid vehicle carrying an effective quantity of active agents, has one of its active agents defined by particles of water insoluble non-toxic thermoplastic resin, which are impregnated with a germicide adequate to be used in oral hygiene.

In the above mentioned composition, the germicidal or bactericidal active agent is maintained aggregated, preferably by hot impregnation, in the particles of water insoluble thermoplastic resin, said particles remaining mechanically retained in spaces of the oral cavity, allowing the progressive release of the germicide impregnated therein. In this solution, the particles of thermoplastic resin present dimensions smaller than 50 microns, preferably from 0,1 to 20 microns, being efficient, regarding the prolongation of the germicidal actuation, only those mechanically retained in the spaces or interstices of the oral cavity. Those particles not retained in the spaces within the oral cavity will be carried by the saliva and therefore will not present a positive effect in extending the germicidal action of said active agent.

It should be further noted that the particles with dimensions measured in microns require spaces with sufficient dimensions to receive and lodge said particles. It is not possible for these particles to be lodged and mechanically retained in micro recesses existing in the mouth mucosa and in the dental arcade presenting dimensions measured in a unit inferior to the micron. Thus, the degree of removal of the non-retained particles is still relatively high.

Another aspect that limits the efficiency of the composition using micro particles of saliva or water insoluble thermoplastic resin results from the mechanism that connects the germicidal active agent to the carrying particles. In the prior solution, the germicidal agent is impregnated in the particles, which are required to have a certain minimal size, in order to allow impregnation to occur and become an effective source for later dispersion of the germicide in the mouth cavity, upon application of the composition. The impregnation of the germicidal agent in the thermoplastic particles, although retaining the active agent, impairs the dispersion thereof when said dispersion is required, making complex to achieve a balance between the velocity of dispersion and the average time of permanence of the particles mechanically retained in the spaces existing in the mouth cavity.

### OBJECTIVE OF THE INVENTION

It is an object of the present invention to provide a composition for oral hygiene of the type considered herein and which leads to a longer and more efficient action of the germicidal active agent in the mouth cavity, allowing considerable reductions to be obtained in the gingival index and in the plaque index in relation to the known compositions.

### SUMMARY OF THE INVENTION

The above mentioned object is achieved by the provision of a composition for oral hygiene, comprising an aqueous vehicle carrying an efficient quantity of a germicidal active agent for oral hygiene.

According to the invention, the germicidal active agent is adsorbed in particles with 80-100 nm of a water insoluble non-toxic polymer, in a quantity of about 1% to 20% by weight of said particles, said polymer presenting a molecular structure with electrochemical positive ends.

### BRIEF DESCRIPTION OF THE DRAWINGS

The invention will be described below, with reference to the enclosed drawings in which:

FIG. 1 illustrates a graph summarizing the results of the reduction of the gingival index in a group of persons submitted to a study with the use of the composition of the present invention; and

FIG. 2 illustrates a graph summarizing the results of the reduction of the plaque index in a group of persons submitted to a study with the use of the composition of the present invention.

According to the invention, the carrying and retaining means, which is specific for the germicidal active agent, is defined by particles or spheres with dimensions ranging from 80 to 100 nm, therefore smaller than those of the prior art, which are used to retain the germicidal active agent by impregnation in the structure thereof.

In the solution now proposed, the particles of the carrying means of the germicidal active agent are obtained from a polymer with a spongy structure and presenting electrochemical positive ends, which are attracted by the electrochemically negative charge existing in the mouth mucosa. The reduced dimension of the particles of the carrying means, together with the electrochemical attraction thereof in relation to the mouth mucosa, allows said particles to be more easily mechanically trapped and electrochemically retained in the spaces of the oral cavity, in which they remain for a longer period of time without being dissolved or carried by the saliva.

Besides the advantage obtained with the increase of both the quantity and the dispersion of the particles of the carrying means that are mechanically retained in the oral cavity, and also with the longer time of retention, the germicidal active agent is adsorbed in the particles of the carrying means, i.e., the molecules of the germicidal active agent are electrochemically affixed to the surface of the spongy structure of the polymer. There is no impregnation of the germicidal active agent in the particles of the polymeric material. Thus, while the particles remain in the oral cavity, the germicidal active agent is gradually released from the spongy and polymeric molecular structure more efficiently and in a certain velocity, which will mostly depend on the structure of the polymer.

Preferably, the polymer used is polysiloxane. Nevertheless, it should be understood that other polymers might be used, having the same characteristics of atoxicity and with the molecular structure presenting electrochemical positive ends. As examples of other polymers, the polymers based on polystyrene and silicones may be mentioned.

As a germicidal active agent to be adsorbed in the particles of the polymeric carrying means, Triclosan is preferably used, in a concentration of 5%, although it is possible to substitute it for other germicidal active agents that may be adsorbed in the molecular structure of the polymeric particles. Examples of other possible germicidal agents are: chlorhexidine, sodium lauril sulfate, cetylpyridinium chloride, benzalkonium chloride, benzethonium chloride and other ammonium quaternaries, zinc chloride and other salts of zinc, eucalyptol, thymol, menthol, methyl salicylate and other essential oils, antibiotics, enzymes and other compounds that may be used as antiseptic agents.

According to the invention, the germicidal active agent is adsorbed in the particles of the polymeric carrying means in a quantity of about 1% to 20% by weight of said particles, the quantity of the latter in the composition being determined in order to provide said composition with about 0.1% to 5% of the germicidal active agent.

In a possible exemplary formulation, the present composition comprises, by weight of an aqueous solution, the following constituents:

a) Antiseptic agent, such as, for example, ethyl alcohol in a quantity from about 0% to 30%.

b) Surfactant (Poloxamer 407 or Pluronic F127) in a concentration from 0.5% to 5.0%.

c) Preservative, methylparaben, in a concentration from 0.01% to 2.0%.

d) Preservative, propylparaben, in a concentration from 0.01% to 2.0%.

e) Anti-caries agent, sodium fluoride, in a concentration from 0.045% to 2.0%.

f) Buffer, monobasic sodium phosphate, in a concentration from 0.01% to 1.0%.

g) Buffer, dibasic sodium phosphate, in a concentration from 0.01% to 1.0%.

h) Sweetening agent, sucrose, in a concentration from 0.01% to 5.0%.

i) Emollient, glycerin, in a concentration from 0.05% to 10%.

j) Antiseptic agent, cetylpyridinium chloride, in a concentration from 0.01% to 5.0%.

k) Antiseptic system consisting of germicide carrying particles, in a concentration from 0.1% to 5.0%.

l) Flavorant (for example, mint T21122-8) in a concentration from 0.1% to 5%.

m) Colorant (Bleu n-1, for example) in a concentration from 0.0005% to 1%.

The study described below, involving twenty-five persons, had the objective to determine the reductions that can be reached in both the gingival index and the plaque index, by using an odontological mouthwash with the composition of the present invention.

The persons submitted to the study had their gingival and plaque indexes evaluated in three distinct moments of the experiment. The first at the beginning of the study, the second fifteen days later, and the third at the end of the study, i.e., thirty days from the beginning.

The evaluation of the bacterial plaque of each person of the study was made by two skilled examiners in the following way:

the teeth were colored with a plaque enhancing solution, before and just after brushing and mouth washing, in the three distinct moments mentioned above, with the scores being registered and tabulated.

The index used to quantify the bacterial plaque existing in said moments of the experiment was that of Quiqley-Hime modified by Turesky, taking into consideration the following values:

0=absence of plaque;

1=isolated spots of plaque along the gingival margin;

2=thin (less than 1 mm) continuous plaque band along the gingival margin;

3=plaque band with more than 1 mm;

4=plaque covering ⅓ or more, but less than ⅔ of the surface;

5=plaque covering ⅔ or more of the surface.

In order to verify the state of the gum, the modified Löe and Silness gingival index was used, since it is less invasive and more sensitive. By this index, the marginal gum, as well as the buccal and lingual papillary gums of each tooth were evaluated according to the method applied for obtaining the bacterial plaque index, said index being however expressed by the following values:

0. absence of inflammation;

1. light inflammation, slight change of color, little alteration in the texture of any portion, but not the whole marginal or papillary gum;

2. light inflammation, slight change of color, little alteration in the texture, but involving the whole marginal or papillary gum;

3. light inflammation, gloss, redness, edema and/or hypertrophia of part of the marginal or papillary gum;

4. light inflammation, gloss, redness, edema and/or hypertrophia of the whole marginal gum or papillary gum;

5. severe inflammation presenting redness, edema and/or hypertrophia of the marginal or papillary gum, spontaneous bleeding, congestion or ulceration.

At the end of fifteen and thirty days, this procedure was repeated, following the same characteristics and method.

The experiment lasted for thirty days and each person used the mouth wash solution three times a day.

The data obtained in each moment of the experiment were compared, and at the end of the experiment a statistical analysis of the T type test was applied.

The gingival index after mouth washing with the present composition for 15 and 30 days was 0.98 and 0.50, respectively (tables 1 and 2):

TABLE 1
Table 1: Gingival index after mouth washing with the
composition containing nano-spheres of Triclosan for
15 days

| | | | | | | | | | GINGIVAL INDEX AFTER MOUTH | | | | | | | |
|---|---|---|---|---|---|---|---|---|---|---|---|---|---|---|---|---|
| | INITIAL GINGIVAL INDEX | | | | | | | | WASHING (15-DAY TEST) | | | | | | | |
| CLIENT | 11 | 16 | 21 | 26 | 31 | 36 | 41 | 46 | 11 | 16 | 21 | 26 | 31 | 36 | 41 | 46 |
| 1 | 2.00 | 5.00 | 1.00 | 5.00 | 3.00 | 3.00 | 2.00 | 3.00 | 0.00 | 0.00 | 0.00 | 3.00 | 2.00 | 3.00 | 0.00 | 3.00 |
| 2 | 2.00 | 2.00 | 5.00 | 2.00 | 3.00 | 5.00 | 3.00 | 5.00 | 0.00 | 0.00 | 0.00 | 2.00 | 0.00 | 0.00 | 0.00 | 0.00 |
| 3 | 5.00 | 5.00 | 2.00 | 5.00 | 4.00 | 3.00 | 3.00 | 3.00 | 0.00 | 0.00 | 0.00 | 2.00 | 4.00 | 3.00 | 2.00 | 0.00 |
| 4 | 5.00 | 5.00 | 5.00 | 5.00 | 5.00 | 3.00 | 0.00 | 0.00 | 3.00 | 3.00 | 0.00 | 0.00 | 0.00 | 0.00 | 1.00 | 0.00 |
| 5 | 2.00 | 4.00 | 5.00 | 4.00 | 2.00 | 3.00 | 2.00 | 3.00 | 0.00 | 0.00 | 0.00 | 3.00 | 2.00 | 3.00 | 0.00 | 3.00 |
| 6 | 5.00 | 3.00 | 4.00 | 3.00 | 5.00 | 5.00 | 3.00 | 5.00 | 0.00 | 0.00 | 0.00 | 2.00 | 0.00 | 0.00 | 0.00 | 0.00 |
| 7 | 5.00 | 3.00 | 3.00 | 3.00 | 5.00 | 2.00 | 3.00 | 3.00 | 0.00 | 0.00 | 0.00 | 2.00 | 4.00 | 3.00 | 2.00 | 0.00 |
| 8 | 4.00 | 4.00 | 3.00 | 4.00 | 4.00 | 2.00 | 5.00 | 5.00 | 3.00 | 3.00 | 0.00 | 0.00 | 0.00 | 0.00 | 1.00 | 0.00 |
| 9 | 3.00 | 4.00 | 4.00 | 4.00 | 3.00 | 3.00 | 2.00 | 3.00 | 0.00 | 0.00 | 0.00 | 3.00 | 2.00 | 3.00 | 0.00 | 3.00 |
| 10 | 3.00 | 3.00 | 4.00 | 3.00 | 3.00 | 5.00 | 3.00 | 5.00 | 0.00 | 0.00 | 0.00 | 2.00 | 0.00 | 0.00 | 0.00 | 0.00 |
| 11 | 4.00 | 3.00 | 3.00 | 3.00 | 4.00 | 2.00 | 3.00 | 3.00 | 0.00 | 0.00 | 0.00 | 2.00 | 4.00 | 3.00 | 2.00 | 0.00 |
| 12 | 4.00 | 1.00 | 3.00 | 1.00 | 4.00 | 2.00 | 5.00 | 5.00 | 3.00 | 3.00 | 0.00 | 0.00 | 0.00 | 0.00 | 1.00 | 0.00 |
| 13 | 3.00 | 3.00 | 1.00 | 3.00 | 3.00 | 3.00 | 2.00 | 3.00 | 0.00 | 0.00 | 0.00 | 3.00 | 2.00 | 3.00 | 0.00 | 3.00 |
| 14 | 3.00 | 3.00 | 3.00 | 3.00 | 3.00 | 5.00 | 3.00 | 5.00 | 0.00 | 0.00 | 0.00 | 2.00 | 0.00 | 0.00 | 0.00 | 0.00 |
| 15 | 1.00 | 2.00 | 3.00 | 2.00 | 1.00 | 3.00 | 3.00 | 3.00 | 0.00 | 0.00 | 0.00 | 2.00 | 4.00 | 3.00 | 2.00 | 0.00 |
| 16 | 3.00 | 4.00 | 2.00 | 4.00 | 3.00 | 3.00 | 5.00 | 5.00 | 3.00 | 3.00 | 0.00 | 0.00 | 0.00 | 0.00 | 1.00 | 0.00 |
| 17 | 3.00 | 4.00 | 4.00 | 4.00 | 3.00 | 3.00 | 2.00 | 3.00 | 0.00 | 0.00 | 0.00 | 3.00 | 2.00 | 3.00 | 0.00 | 3.00 |
| 18 | 2.00 | 3.00 | 4.00 | 3.00 | 2.00 | 5.00 | 3.00 | 5.00 | 0.00 | 0.00 | 0.00 | 2.00 | 0.00 | 0.00 | 0.00 | 0.00 |
| 19 | 4.00 | 4.00 | 3.00 | 4.00 | 4.00 | 2.00 | 3.00 | 3.00 | 0.00 | 0.00 | 0.00 | 2.00 | 4.00 | 3.00 | 2.00 | 0.00 |
| 20 | 4.00 | 4.00 | 4.00 | 4.00 | 4.00 | 2.00 | 5.00 | 5.00 | 3.00 | 3.00 | 0.00 | 0.00 | 0.00 | 0.00 | 1.00 | 0.00 |
| 21 | 3.00 | 4.00 | 4.00 | 4.00 | 3.00 | 3.00 | 2.00 | 3.00 | 0.00 | 0.00 | 0.00 | 3.00 | 2.00 | 3.00 | 0.00 | 3.00 |
| 22 | 4.00 | 2.00 | 4.00 | 2.00 | 4.00 | 5.00 | 3.00 | 5.00 | 0.00 | 0.00 | 0.00 | 2.00 | 0.00 | 0.00 | 0.00 | 0.00 |
| 23 | 4.00 | 5.00 | 2.00 | 5.00 | 4.00 | 2.00 | 3.00 | 3.00 | 0.00 | 0.00 | 0.00 | 2.00 | 4.00 | 3.00 | 2.00 | 0.00 |
| 24 | 4.00 | 4.00 | 5.00 | 2.00 | 4.00 | 3.00 | 5.00 | 5.00 | 3.00 | 3.00 | 0.00 | 0.00 | 0.00 | 0.00 | 1.00 | 0.00 |
| 25 | 2.00 | 1.00 | 1.00 | 1.00 | 2.00 | 3.00 | 2.00 | 3.00 | 0.00 | 0.00 | 0.00 | 3.00 | 2.00 | 3.00 | 0.00 | 3.00 |
| AVERAGE | 3.36 | 3.40 | 3.28 | 3.32 | 3.40 | 3.20 | 3.00 | 3.76 | 0.72 | 0.72 | 0.00 | 1.80 | 1.52 | 1.56 | 0.72 | 0.84 |

Initial gingival index: 3.34
Gingival index after mouth washing for 15 days: 0.98

TABLE 2
Table 2: Gingival index after mouth washing for 30 days

| | | | | | | | | | GINGIVAL INDEX AFTER MOUTH | | | | | | | |
|---|---|---|---|---|---|---|---|---|---|---|---|---|---|---|---|---|
| | INITIAL GINGIVAL INDEX | | | | | | | | WASHING (30-DAY TEST) | | | | | | | |
| CLIENT | 11 | 16 | 21 | 26 | 31 | 36 | 41 | 46 | 11 | 16 | 21 | 26 | 31 | 36 | 41 | 46 |
| 1 | 2.00 | 5.00 | 1.00 | 5.00 | 3.00 | 3.00 | 2.00 | 3.00 | 0.00 | 0.00 | 0.00 | 0.00 | 2.00 | 0.00 | 2.00 | 0.00 |
| 2 | 2.00 | 2.00 | 5.00 | 2.00 | 3.00 | 5.00 | 3.00 | 5.00 | 0.00 | 3.00 | 0.00 | 0.00 | 0.00 | 1.00 | 0.00 | 2.00 |
| 3 | 5.00 | 5.00 | 2.00 | 5.00 | 4.00 | 3.00 | 3.00 | 3.00 | 0.00 | 0.00 | 0.00 | 0.00 | 0.00 | 0.00 | 0.00 | 0.00 |
| 4 | 5.00 | 5.00 | 5.00 | 5.00 | 5.00 | 3.00 | 0.00 | 0.00 | 0.00 | 2.00 | 0.00 | 2.00 | 0.00 | 0.00 | 0.00 | 2.00 |
| 5 | 2.00 | 4.00 | 5.00 | 4.00 | 2.00 | 3.00 | 2.00 | 3.00 | 0.00 | 0.00 | 0.00 | 0.00 | 2.00 | 0.00 | 2.00 | 0.00 |
| 6 | 5.00 | 3.00 | 4.00 | 3.00 | 5.00 | 5.00 | 3.00 | 5.00 | 0.00 | 3.00 | 0.00 | 0.00 | 0.00 | 1.00 | 0.00 | 2.00 |
| 7 | 5.00 | 3.00 | 3.00 | 3.00 | 5.00 | 2.00 | 3.00 | 3.00 | 0.00 | 0.00 | 0.00 | 0.00 | 0.00 | 0.00 | 0.00 | 0.00 |
| 8 | 4.00 | 4.00 | 3.00 | 4.00 | 4.00 | 2.00 | 5.00 | 5.00 | 0.00 | 2.00 | 0.00 | 2.00 | 0.00 | 0.00 | 0.00 | 2.00 |
| 9 | 3.00 | 4.00 | 4.00 | 4.00 | 3.00 | 3.00 | 2.00 | 3.00 | 0.00 | 0.00 | 0.00 | 0.00 | 2.00 | 0.00 | 2.00 | 0.00 |
| 10 | 3.00 | 3.00 | 4.00 | 3.00 | 3.00 | 5.00 | 3.00 | 5.00 | 0.00 | 3.00 | 0.00 | 0.00 | 0.00 | 1.00 | 0.00 | 2.00 |
| 11 | 4.00 | 3.00 | 3.00 | 3.00 | 4.00 | 2.00 | 3.00 | 3.00 | 0.00 | 0.00 | 0.00 | 0.00 | 0.00 | 0.00 | 0.00 | 0.00 |
| 12 | 4.00 | 1.00 | 3.00 | 1.00 | 4.00 | 2.00 | 5.00 | 5.00 | 0.00 | 2.00 | 0.00 | 2.00 | 0.00 | 0.00 | 0.00 | 2.00 |
| 13 | 3.00 | 3.00 | 1.00 | 3.00 | 3.00 | 3.00 | 2.00 | 3.00 | 0.00 | 0.00 | 0.00 | 0.00 | 2.00 | 0.00 | 2.00 | 0.00 |
| 14 | 3.00 | 3.00 | 3.00 | 3.00 | 3.00 | 5.00 | 3.00 | 5.00 | 0.00 | 3.00 | 0.00 | 0.00 | 0.00 | 1.00 | 0.00 | 2.00 |
| 15 | 1.00 | 2.00 | 3.00 | 2.00 | 1.00 | 3.00 | 3.00 | 3.00 | 0.00 | 0.00 | 0.00 | 0.00 | 0.00 | 0.00 | 0.00 | 0.00 |
| 16 | 3.00 | 4.00 | 2.00 | 4.00 | 3.00 | 3.00 | 5.00 | 5.00 | 0.00 | 2.00 | 0.00 | 2.00 | 0.00 | 0.00 | 0.00 | 2.00 |
| 17 | 3.00 | 4.00 | 4.00 | 4.00 | 3.00 | 3.00 | 2.00 | 3.00 | 0.00 | 0.00 | 0.00 | 0.00 | 2.00 | 0.00 | 2.00 | 0.00 |
| 18 | 2.00 | 3.00 | 4.00 | 3.00 | 2.00 | 5.00 | 3.00 | 5.00 | 0.00 | 3.00 | 0.00 | 0.00 | 0.00 | 1.00 | 0.00 | 2.00 |
| 19 | 4.00 | 4.00 | 3.00 | 4.00 | 4.00 | 2.00 | 3.00 | 3.00 | 0.00 | 0.00 | 0.00 | 0.00 | 0.00 | 0.00 | 0.00 | 0.00 |
| 20 | 4.00 | 4.00 | 4.00 | 4.00 | 4.00 | 2.00 | 5.00 | 5.00 | 0.00 | 2.00 | 0.00 | 2.00 | 0.00 | 0.00 | 0.00 | 2.00 |
| 21 | 3.00 | 4.00 | 4.00 | 4.00 | 3.00 | 3.00 | 2.00 | 3.00 | 0.00 | 0.00 | 0.00 | 0.00 | 2.00 | 0.00 | 2.00 | 0.00 |
| 22 | 4.00 | 2.00 | 4.00 | 2.00 | 4.00 | 5.00 | 3.00 | 5.00 | 0.00 | 3.00 | 0.00 | 0.00 | 0.00 | 1.00 | 0.00 | 2.00 |
| 23 | 4.00 | 5.00 | 2.00 | 5.00 | 4.00 | 2.00 | 3.00 | 3.00 | 0.00 | 0.00 | 0.00 | 0.00 | 0.00 | 0.00 | 0.00 | 0.00 |
| 24 | 4.00 | 4.00 | 5.00 | 2.00 | 4.00 | 3.00 | 5.00 | 5.00 | 0.00 | 2.00 | 0.00 | 2.00 | 0.00 | 0.00 | 0.00 | 2.00 |
| 25 | 2.00 | 1.00 | 1.00 | 1.00 | 2.00 | 3.00 | 2.00 | 3.00 | 0.00 | 0.00 | 0.00 | 0.00 | 2.00 | 0.00 | 2.00 | 0.00 |
| AVERAGE | 3.36 | 3.40 | 3.28 | 3.32 | 3.40 | 3.20 | 3.00 | 3.76 | 0.00 | 1.20 | 0.00 | 0.48 | 0.56 | 0.24 | 0.56 | 0.96 |

Initial gingival index: 3.34
Gingival index after mouth washing for 30 days: 0.5
Reduction of the index at the end of 30 days: 2.84

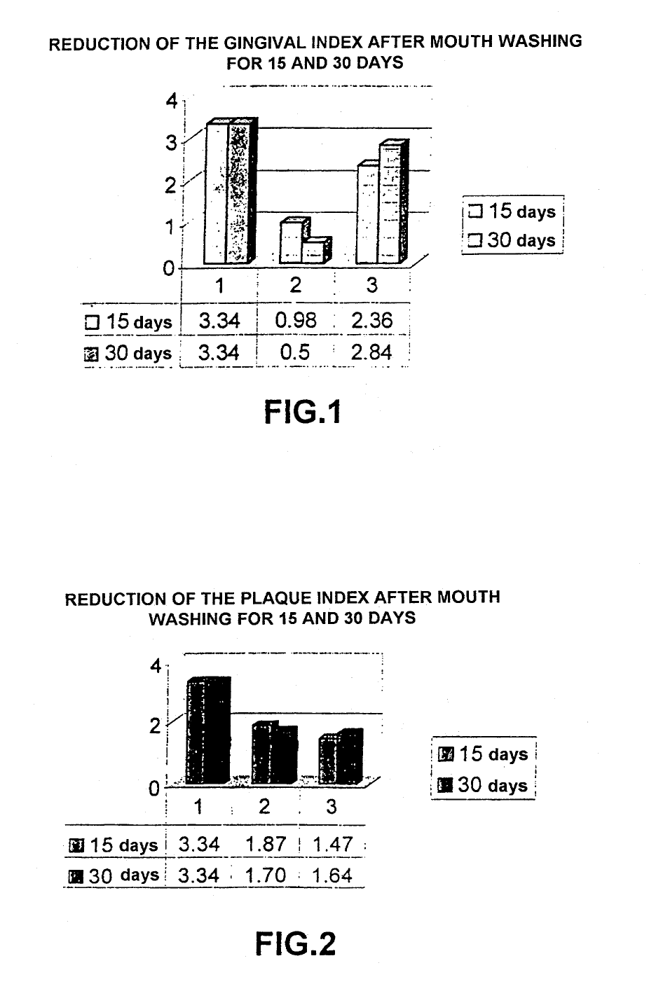

These results demonstrate a reduction in the gingival index of 85% at the end of 30 days since the beginning of the study, being summarized in the graph of FIG. 1. The plaque index after mouth washing with the present composition for 15 and for 30 days was 1.87 and 1.70, respectively (tables 3 and 4).

TABLE 3
Table 3: Quantity of plaque removed, expressed by
Turesky index after mouth washing for 15 days

| | | | | | | | | | GINGIVAL INDEX AFTER MOUTH | | | | | | | |
|---|---|---|---|---|---|---|---|---|---|---|---|---|---|---|---|---|
| | INITIAL GINGIVAL INDEX | | | | | | | | WASHING (30-DAY TEST) | | | | | | | |
| CLIENT | 11 | 16 | 21 | 26 | 31 | 36 | 41 | 46 | 11 | 16 | 21 | 26 | 31 | 36 | 41 | 46 |
| 1 | 3.00 | 2.00 | 3.00 | 3.00 | 3.00 | 3.00 | 2.00 | 3.00 | 1.00 | 4.00 | 1.00 | 1.00 | 1.00 | 2.00 | 2.00 | 1.00 |
| 2 | 3.00 | 3.00 | 3.00 | 3.00 | 3.00 | 3.00 | 3.00 | 3.00 | 2.00 | 0.00 | 1.00 | 1.00 | 1.00 | 2.00 | 0.00 | 2.00 |
| 3 | 4.00 | 3.00 | 4.00 | 4.00 | 4.00 | 2.00 | 3.00 | 4.00 | 2.00 | 2.00 | 2.00 | 2.00 | 2.00 | 2.00 | 0.00 | 2.00 |
| 4 | 5.00 | 2.00 | 5.00 | 5.00 | 5.00 | 5.00 | 2.00 | 5.00 | 1.00 | 2.00 | 0.00 | 2.00 | 2.00 | 0.00 | 2.00 | 3.00 |
| 5 | 3.00 | 2.00 | 3.00 | 3.00 | 3.00 | 3.00 | 2.00 | 3.00 | 1.00 | 4.00 | 3.00 | 3.00 | 1.00 | 3.00 | 2.00 | 3.00 |
| 6 | 3.00 | 2.00 | 3.00 | 3.00 | 3.00 | 3.00 | 2.00 | 3.00 | 1.00 | 4.00 | 3.00 | 3.00 | 1.00 | 3.00 | 2.00 | 3.00 |
| 7 | 3.00 | 3.00 | 3.00 | 3.00 | 3.00 | 3.00 | 3.00 | 3.00 | 2.00 | 0.00 | 3.00 | 3.00 | 1.00 | 3.00 | 0.00 | 2.00 |
| 8 | 5.00 | 2.00 | 5.00 | 5.00 | 5.00 | 5.00 | 2.00 | 5.00 | 2.00 | 0.00 | 3.00 | 3.00 | 1.00 | 3.00 | 0.00 | 2.00 |
| 9 | 4.00 | 3.00 | 4.00 | 4.00 | 4.00 | 4.00 | 3.00 | 4.00 | 2.00 | 0.00 | 3.00 | 3.00 | 1.00 | 3.00 | 2.00 | 2.00 |
| 10 | 3.00 | 2.00 | 3.00 | 3.00 | 3.00 | 3.00 | 2.00 | 3.00 | 2.00 | 2.00 | 2.00 | 2.00 | 2.00 | 3.00 | 2.00 | 2.00 |
| 11 | 3.00 | 3.00 | 3.00 | 3.00 | 3.00 | 3.00 | 3.00 | 3.00 | 2.00 | 0.00 | 1.00 | 1.00 | 1.00 | 3.00 | 2.00 | 2.00 |
| 12 | 3.00 | 2.00 | 3.00 | 3.00 | 3.00 | 3.00 | 2.00 | 3.00 | 1.00 | 4.00 | 1.00 | 1.00 | 1.00 | 3.00 | 2.00 | 3.00 |
| 13 | 4.00 | 3.00 | 4.00 | 4.00 | 4.00 | 4.00 | 3.00 | 4.00 | 2.00 | 0.00 | 1.00 | 1.00 | 1.00 | 3.00 | 0.00 | 2.00 |
| 14 | 3.00 | 3.00 | 3.00 | 3.00 | 3.00 | 3.00 | 3.00 | 3.00 | 1.00 | 4.00 | 1.00 | 1.00 | 1.00 | 3.00 | 2.00 | 1.00 |
| 15 | 4.00 | 3.00 | 4.00 | 4.00 | 4.00 | 4.00 | 2.00 | 4.00 | 2.00 | 2.00 | 2.00 | 2.00 | 2.00 | 3.00 | 0.00 | 2.00 |
| 16 | 4.00 | 3.00 | 4.00 | 4.00 | 4.00 | 4.00 | 2.00 | 4.00 | 1.00 | 2.00 | 0.00 | 3.00 | 2.00 | 0.00 | 2.00 | 3.00 |
| 17 | 3.00 | 2.00 | 5.00 | 5.00 | 5.00 | 5.00 | 2.00 | 5.00 | 1.00 | 2.00 | 0.00 | 3.00 | 2.00 | 0.00 | 2.00 | 3.00 |
| 18 | 3.00 | 2.00 | 3.00 | 3.00 | 3.00 | 3.00 | 2.00 | 3.00 | 2.00 | 2.00 | 2.00 | 3.00 | 2.00 | 2.00 | 3.00 | 3.00 |
| 19 | 5.00 | 2.00 | 5.00 | 5.00 | 5.00 | 5.00 | 2.00 | 4.00 | 1.00 | 2.00 | 0.00 | 3.00 | 2.00 | 0.00 | 2.00 | 3.00 |
| 20 | 3.00 | 3.00 | 3.00 | 3.00 | 3.00 | 3.00 | 3.00 | 3.00 | 2.00 | 2.00 | 2.00 | 3.00 | 2.00 | 2.00 | 3.00 | 3.00 |
| 21 | 3.00 | 3.00 | 3.00 | 3.00 | 3.00 | 3.00 | 3.00 | 3.00 | 1.00 | 2.00 | 0.00 | 3.00 | 2.00 | 0.00 | 2.00 | 3.00 |
| 22 | 3.00 | 2.00 | 4.00 | 4.00 | 4.00 | 4.00 | 3.00 | 4.00 | 1.00 | 2.00 | 0.00 | 3.00 | 2.00 | 0.00 | 2.00 | 3.00 |
| 23 | 4.00 | 2.00 | 5.00 | 3.00 | 5.00 | 5.00 | 2.00 | 5.00 | 2.00 | 2.00 | 2.00 | 3.00 | 2.00 | 2.00 | 3.00 | 3.00 |
| 24 | 3.00 | 3.00 | 3.00 | 3.00 | 3.00 | 3.00 | 3.00 | 3.00 | 2.00 | 2.00 | 2.00 | 3.00 | 2.00 | 2.00 | 3.00 | 3.00 |
| 25 | 5.00 | 2.00 | 5.00 | 5.00 | 3.00 | 4.00 | 2.00 | 4.00 | 1.00 | 4.00 | 1.00 | 3.00 | 1.00 | 2.00 | 2.00 | 3.00 |
| AVERAGE | 3.56 | 2.48 | 3.72 | 3.64 | 3.64 | 3.60 | 2.44 | 3.64 | 1.52 | 2.00 | 1.44 | 2.36 | 1.52 | 1.96 | 1.68 | 2.48 |

Initial gingival index: 3.34
Gingival index after mouth washing for 15 days: 1.87
Removal of the median bacterial plaque according to Turesky index: 1.47

TABLE 4
Table 4: Quantity of plaque removed, expressed by
Turesky index, after mouth washing for 30 days

| | | | | | | | | | GINGIVAL INDEX AFTER MOUTH | | | | | | | |
|---|---|---|---|---|---|---|---|---|---|---|---|---|---|---|---|---|
| | INITIAL GINGIVAL INDEX | | | | | | | | WASHING (30-DAY TEST) | | | | | | | |
| CLIENT | 11 | 16 | 21 | 26 | 31 | 36 | 41 | 46 | 11 | 16 | 21 | 26 | 31 | 36 | 41 | 46 |
| 1 | 3.00 | 2.00 | 3.00 | 3.00 | 3.00 | 3.00 | 2.00 | 3.00 | 0.00 | 2.00 | 0.00 | 1.00 | 0.00 | 2.00 | 0.00 | 1.00 |
| 2 | 3.00 | 3.00 | 3.00 | 3.00 | 3.00 | 3.00 | 3.00 | 3.00 | 0.00 | 2.00 | 3.00 | 1.00 | 1.00 | 3.00 | 2.00 | 2.00 |
| 3 | 4.00 | 3.00 | 4.00 | 4.00 | 4.00 | 2.00 | 3.00 | 4.00 | 2.00 | 1.00 | 0.00 | 0.00 | 1.00 | 3.00 | 0.00 | 1.00 |
| 4 | 5.00 | 2.00 | 5.00 | 5.00 | 5.00 | 5.00 | 2.00 | 5.00 | 0.00 | 1.00 | 0.00 | 0.00 | 1.00 | 3.00 | 3.00 | 3.00 |
| 5 | 3.00 | 2.00 | 3.00 | 3.00 | 3.00 | 3.00 | 2.00 | 3.00 | 0.00 | 3.00 | 3.00 | 0.00 | 3.00 | 3.00 | 3.00 | 1.00 |
| 6 | 3.00 | 2.00 | 3.00 | 3.00 | 3.00 | 3.00 | 2.00 | 3.00 | 0.00 | 3.00 | 3.00 | 3.00 | 3.00 | 0.00 | 3.00 | 3.00 |
| 7 | 3.00 | 3.00 | 3.00 | 3.00 | 3.00 | 3.00 | 3.00 | 3.00 | 0.00 | 3.00 | 3.00 | 0.00 | 3.00 | 3.00 | 0.00 | 1.00 |
| 8 | 5.00 | 2.00 | 5.00 | 5.00 | 5.00 | 5.00 | 2.00 | 5.00 | 2.00 | 3.00 | 3.00 | 3.00 | 3.00 | 3.00 | 0.00 | 1.00 |
| 9 | 4.00 | 3.00 | 4.00 | 4.00 | 4.00 | 4.00 | 3.00 | 4.00 | 2.00 | 3.00 | 3.00 | 3.00 | 3.00 | 3.00 | 1.00 | 3.00 |
| 10 | 3.00 | 2.00 | 3.00 | 3.00 | 3.00 | 3.00 | 2.00 | 3.00 | 0.00 | 2.00 | 0.00 | 1.00 | 3.00 | 3.00 | 0.00 | 1.00 |
| 11 | 3.00 | 3.00 | 3.00 | 3.00 | 3.00 | 3.00 | 3.00 | 3.00 | 2.00 | 2.00 | 3.00 | 1.00 | 3.00 | 3.00 | 2.00 | 2.00 |
| 12 | 3.00 | 2.00 | 3.00 | 3.00 | 3.00 | 3.00 | 2.00 | 3.00 | 0.00 | 1.00 | 0.00 | 3.00 | 3.00 | 3.00 | 3.00 | 1.00 |
| 13 | 4.00 | 3.00 | 4.00 | 4.00 | 4.00 | 4.00 | 3.00 | 4.00 | 2.00 | 1.00 | 0.00 | 3.00 | 3.00 | 3.00 | 1.00 | 3.00 |
| 14 | 3.00 | 3.00 | 3.00 | 3.00 | 3.00 | 3.00 | 3.00 | 3.00 | 2.00 | 2.00 | 0.00 | 1.00 | 3.00 | 2.00 | 0.00 | 1.00 |
| 15 | 4.00 | 3.00 | 4.00 | 4.00 | 4.00 | 4.00 | 2.00 | 4.00 | 0.00 | 2.00 | 0.00 | 1.00 | 3.00 | 2.00 | 0.00 | 1.00 |
| 16 | 4.00 | 3.00 | 4.00 | 4.00 | 4.00 | 4.00 | 2.00 | 4.00 | 0.00 | 1.00 | 1.00 | 3.00 | 1.00 | 3.00 | 3.00 | 3.00 |
| 17 | 3.00 | 2.00 | 5.00 | 5.00 | 5.00 | 5.00 | 2.00 | 5.00 | 2.00 | 1.00 | 1.00 | 0.00 | 1.00 | 3.00 | 3.00 | 1.00 |
| 18 | 3.00 | 2.00 | 3.00 | 3.00 | 3.00 | 3.00 | 2.00 | 3.00 | 2.00 | 2.00 | 1.00 | 1.00 | 1.00 | 3.00 | 2.00 | 2.00 |
| 19 | 5.00 | 2.00 | 5.00 | 5.00 | 5.00 | 5.00 | 2.00 | 4.00 | 2.00 | 1.00 | 1.00 | 3.00 | 1.00 | 3.00 | 2.00 | 3.00 |
| 20 | 3.00 | 3.00 | 3.00 | 3.00 | 3.00 | 3.00 | 3.00 | 3.00 | 0.00 | 2.00 | 1.00 | 1.00 | 0.00 | 3.00 | 2.00 | 1.00 |
| 21 | 3.00 | 3.00 | 3.00 | 3.00 | 3.00 | 3.00 | 3.00 | 3.00 | 2.00 | 3.00 | 1.00 | 1.00 | 1.00 | 3.00 | 2.00 | 2.00 |
| 22 | 3.00 | 2.00 | 4.00 | 4.00 | 4.00 | 4.00 | 3.00 | 4.00 | 2.00 | 2.00 | 1.00 | 1.00 | 0.00 | 2.00 | 2.00 | 1.00 |
| 23 | 4.00 | 2.00 | 5.00 | 3.00 | 5.00 | 5.00 | 2.00 | 5.00 | 3.00 | 3.00 | 1.00 | 1.00 | 1.00 | 3.00 | 2.00 | 2.00 |
| 24 | 3.00 | 3.00 | 3.00 | 3.00 | 3.00 | 3.00 | 3.00 | 3.00 | 2.00 | 2.00 | 1.00 | 1.00 | 0.00 | 3.00 | 2.00 | 1.00 |
| 25 | 5.00 | 2.00 | 5.00 | 5.00 | 3.00 | 4.00 | 2.00 | 4.00 | 3.00 | 3.00 | 1.00 | 1.00 | 1.00 | 3.00 | 2.00 | 2.00 |
| AVERAGE | 3.56 | 2.48 | 3.72 | 3.64 | 3.64 | 3.60 | 2.44 | 3.64 | 1.20 | 2.04 | 1.24 | 1.36 | 1.72 | 2.72 | 1.60 | 1.72 |

Initial gingival index: 3.34
Gingival index after mouth washing for 30 days: 1.70
Removal of the median bacterial plaque according to Turesky index: 1.64

These results demonstrate a reduction in the plaque index of 49%, at the end of 30 days since the beginning of the study, summarized in the graph of FIG. 2.

In the conditions of the experiment, mouth washing with the composition of the present invention caused a reduction of 85% in the gingival index and of 49% in the plaque index, these results being highly superior to those known, situated within the range of 55% and 45%, respectively.

When comparing the results achieved by the evaluated products, it is verified that using the mouth wash with Triclosan in nano-spheres reduces the quantity of plaque 4% more than using the Triclosan-based mouth wash. This difference is more relevant when taking into consideration the gingival index, when such difference is 30%, which makes the result statistically significant, with the value p<0.05.

1. A composition for oral hygiene, comprising an aqueous vehicle carrying an efficient quantity of a germicidal active agent for oral hygiene, characterized in that the germicidal active agent is adsorbed in particles with 80-100 nm of a water insoluble non-toxic polymer, in a quantity of about 1% to 20% by weight of said particles, said polymer presenting a spongy molecular structure with electrochemical positive ends.

2. Composition, according to claim 1, characterized in that the particles are present in a quantity that results in about 0.1% to 5% of the germicidal active agent in the composition.

3. Composition, according to claim 1, characterized in that the water insoluble polymer presents a density of about 0.5 to 2.5 g/cm³.

4. Composition, according to any of the claims 1, 2 or 3, characterized in that the water insoluble polymer is polysiloxane.

5. Composition, according to any of the claims 1, 2, 3 or 4, characterized in that the germicidal active agent is Triclosan at 5%.

6. Composition, according to claim 1, characterized in that it comprises by weight of an aqueous solution, the following constituents:
ethyl alcohol: 9.5%
pluronic F127: 1.75%
methylparaben: 0.20%
propylparaben: 0.02%
sodium fluoride: 0.05%
monobasic sodium phosphate: 0.05%
dibasic sodium phosphate: 0.09%
sucrose: 1.00%
cetylpyridinium chloride: 0.05%
germicide carrying particles: 0.8%
flavorant, mint T21122-8: 0.30%
colorant, Bleu n-1: 0.00065%
